(19) United States
(12) Patent Application Publication
(10) Pub. No.: US 2006/0263321 A1
(43) Pub. Date: Nov. 23, 2006

(54) Regulation of mammalian keratinous tissue using personal care compositions comprising diethylhexyl syringylidene malonate

(75) Inventor: Donald Lynn Bissett, Hamilton, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company

(21) Appl. No.: 11/406,477

(22) Filed: Apr. 18, 2006

Related U.S. Application Data
(60) Provisional application No. 60/681,626, filed on May 17, 2005.

(51) Int. Cl. A61K 8/97; A61K 8/73

(52) U.S. Cl. 424/70.13; 424/74

(57) ABSTRACT

Personal care compositions containing diethylhexyl syringylidene malonate are provided. Methods for regulating the condition of mammalian keratinous tissue by topically applying the personal care compositions are also provided.

### CROSS REFERENCE TO RELATED APPLICATION

This application claims the benefit of U.S. Provisional Patent Application Ser. No. 60/681,626, filed May 17, 2005.

### TECHNICAL FIELD

The present invention relates to personal care compositions containing diethylhexyl syringylidene malonate. The compositions are useful for regulating the condition of mammalian keratinous tissue needing such treatments, particularly for preventing, retarding, and/or treating uneven skin tone, such as, for example, acting as a lightening or pigmentation reduction cosmetic agent.

### BACKGROUND OF THE INVENTION

Currently, there are a number of personal care products that are available to consumers, which are directed toward improving the health and physical appearance of keratinous tissues such as the skin, hair, and nails. The majority of these products are directed to delaying, minimizing or even eliminating skin wrinkling and histological changes typically associated with the aging of skin or environmental damage to human skin. However, there also exists a need for cosmetic agents to prevent, retard, and/or treat uneven skin tone by acting as a lightening or pigmentation reduction cosmetic agent.

Mammalian keratinous tissue, particularly human skin and hair, is subjected to a variety of insults by both extrinsic and intrinsic factors. Such extrinsic factors include ultraviolet radiation, environmental pollution, wind, heat, infrared radiation, low humidity, harsh surfactants, abrasives, etc. Intrinsic factors, on the other hand, include chronological aging and other biochemical changes from within the skin. Whether extrinsic or intrinsic, these factors result in visible signs of skin damage. Typical skin damage includes thinning of the skin, which occurs naturally as one ages. With such thinning, there is a reduction in the cells and blood vessels that supply the skin as well as a flattening of the dermal-epidermal junction that results in weaker mechanical resistance of this junction. See, for example, Oikarinen, “The Aging of Skin: Chronoaging Versus Photoaging,” Photodermatol. Photoimmunol. Photomed., vol. 7, pp. 3-4, 1990. Other damages or changes seen in aging or damaged skin include fine lines, wrinkling, hyperpigmentation, sallowness, sagging, dark under-eye circles, puffy eyes, enlarged pores, diminished rate of turnover, and abnormal desquamation or exfoliation. Additional damage incurred as a result of both external and internal factors includes visible dead skin (i.e., flaking, scaling, dryness, roughness). For hair, these extrinsic and intrinsic factors can contribute to, among other problems, hair bleaching, split ends, fragility, roughness, hair loss, reduction in hair growth rate, and the like. Therefore, there is a need for products and methods that seek to remedy these keratinous tissue conditions.

### SUMMARY OF PREFERRED EMBODIMENTS

Applicants have discovered that personal care compositions that contain certain skin and hair care actives may be used to provide prophylactic as well as therapeutic treatments for keratinous tissue conditions, particularly skin-lightening. In accordance with one preferred embodiment of the present invention, there has now been provided a personal care composition comprising diethylhexyl syringylidene malonate; at least one additional skin and/or hair care active selected from the group consisting of tetrahydrocurcumin, sugar amines, peptides, phytosterol, dialkanoyl hydroxyproline, hexamidine compounds, cetyl pyridinium chloride, N-acyl amino acid compounds, hesperedin, mustard seed extract, glycyrrhizic acid, glycyrrhetinic acid, ergothioneine, melanostatine, sterol esters, idebenone, dehydroacetic acid, Licohalcone A, creatine, creatinine, fever few extract, yeast extract, beta glucans, alpha glucans, their salts, their derivatives, their precursors, and/or combinations thereof; and a dermatologically acceptable carrier. In one embodiment, the personal care composition is not marketed as a sunscreen product (although it may contain sunscreen actives).

The present invention also relates to articles of commerce comprising personal care compositions disclosed herein. In accordance with one preferred embodiment, there has now been provided an article of commerce comprising a personal care composition comprising diethylhexyl syringylidene malonate; and at least one of packaging for the personal care composition and advertisement material pertaining to the personal care composition comprising indicia and/or an image which communicates that the personal care composition can be used in conjunction with an energy delivery device for regulating the condition of mammalian keratinous tissue.

The invention further relates to methods for regulating the condition of mammalian keratinous tissue wherein the methods each comprise the step of topically applying to the keratinous tissue of a mammal needing such treatment, a safe and effective amount of a personal care composition of the invention. In accordance with one preferred embodiment, there has now been provided a method comprising the steps of topically applying a personal care composition comprising diethylhexyl syringylidene malonate to a desired area of tissue; and thereafter applying a second personal care composition comprising a sunscreen active to the desired area of tissue.

In accordance with another preferred embodiment, there has now been provided a method comprising the steps of topically applying a personal care composition comprising diethylhexyl syringylidene malonate to a desired area of tissue; and applying energy to the area of tissue via an energy delivery device.

### DETAILED DESCRIPTION OF ILLUSTRATIVE AND PREFERRED EMBODIMENTS

All percentages and ratios used herein are by weight of the total composition and all measurements made are at 25° C., unless otherwise designated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

The term “keratinous tissue,” as used herein, refers to keratin-containing layers disposed as the outermost protective covering of mammals which includes, but is not limited to, skin, hair, toenails, fingernails, cuticles, hooves, etc.

The term “topical application”, as used herein, means to apply or spread the compositions of the present invention onto the surface of the keratinous tissue.

The term “dermatologically acceptable,” as used herein, means that the compositions or components described are suitable for use in contact with human keratinous tissue without undue toxicity, incompatibility, instability, allergic response, and the like.

The term “safe and effective amount” as used herein means an amount of a compound or composition sufficient to significantly induce a positive benefit, preferably a positive keratinous tissue appearance or feel benefit, including independently or in combination the benefits disclosed herein, but low enough to avoid serious side effects (i.e., to provide a reasonable benefit to risk ratio, within the scope of sound judgment of the skilled artisan).

The term “post-inflammatory hyperpigmentation” as used herein refers to the changes in melanin content as a response to an inflammatory event (e.g., acne, scratch, insect sting or bite, sunburn, etc), especially in dark skin subjects.

The term “hyperpigmentation” as used herein refers to an area of skin wherein the pigmentation is greater than that of an adjacent area of skin (e.g., a pigment spot, an age spot, and the like).

The terms “desquamation, exfoliation, and/or turnover” as used herein mean the removal of the upper layers of the stratum comeum (comprising the horny layers).

The terms “oily and/or shiny appearance” as used herein mean the glossy look mammalian skin tends to exhibit upon the excretion of oil, sebum, and/or sweat from the respective source gland.

The term “sagging” as used herein means the laxity, slackness, or the like condition of skin that occurs as a result of loss of, damage to, alterations to, and/or abnormalities in dermal elastin.

The term “smoothing” and “softening” as used herein means altering the surface of the keratinous tissue such that its tactile feel is improved.

The term “sallowness” as used herein means the pale color, yellow color or the like condition of skin that occurs as a result of a loss of; damage to, alterations to, and/or abnormalities in skin components such that they become colored (e.g., yellow in color) due to processes such as protein glycation and accumulation of lipofuscin or in the decrease in peripheral blood flow that typically accompanies skin aging.

The compositions of the present invention are useful for topical application and for regulating keratinous tissue condition. Regulation of keratinous tissue condition, especially human skin condition, is often required due to conditions that may be induced or caused by factors internal and/or external to the body. For instance, “regulating skin condition” includes prophylactically regulating and/or therapeutically regulating skin condition, and may involve one or more of the following benefits: thickening (i.e., building the epidermis and/or dermis layers of the skin and/or the subcutaneous layers such as fat and muscle and where applicable the keratinous layers of the nail and hair shaft) to reduce atrophy (e.g., of the skin), increasing the convolution of the dermal-epidermal border, non-melanin skin discoloration such as under eye circles, blotching (e.g., uneven red coloration due to, e.g., rosacea) (hereinafter referred to as “red blotchiness”), sallowness (pale or yellow color), discoloration caused by telangiectasia or spider vessels, discolorations due to melanin (e.g., pigment spots, age spots, uneven pigmentation) and other chromophores in the skin (e.g., lipofuscin, protein crosslinks such as those that occur with glycation, and the like). As used herein, prophylactically regulating skin condition includes delaying, minimizing and/or preventing visible and/or tactile discontinuities in skin (e.g., texture irregularities, fine lines, wrinkles, sagging, stretch marks, cellulite, puffy eyes, and the like in the skin which may be detected visually or by feel). As used herein, therapeutically regulating skin condition includes ameliorating (e.g., diminishing, minimizing and/or effacing) discontinuities in skin. Regulating skin condition involves improving skin appearance and/or feel.

As used herein, “regulating skin condition” is intended to include regulation of such signs irrespective of the mechanism of origin.

The compositions of the present invention, including the essential and optional components thereof, are described in detail hereinafter.

### Personal Care Composition

Components

The compositions of the present invention comprise a safe and effective amount of diethylhexyl syringylidene malonate. Diethylhexyl syringylidene malonate is preferably present in an amount of from about 0.01% to about 20% by weight of the composition, more preferably from about 0.1% to about 15% by weight of the composition, even more preferably from about 0.5% to about 10% by weight of the composition.

A preferred diethylhexyl syrinylidene malonate is Oxynex ST® which exhibits anti-oxidant properties. It is available from Rona/Merck.

Additional Skin and/or Hair Care Actives

Compositions of the present invention typically comprise a safe and effective amount of at least one additional skin and/or hair care active. A representative, non-limiting list of such actives includes sugar amines, vitamin B₃, retinoids, hydroquinone, peptides, famesol, phytosterol, dialkanoyl hydroxyproline, hexamidine, salicylic acid, N-acyl amino acid compounds, sunscreen actives, water soluble vitamins, oil soluble vitamins, hesperedin, mustard seed extract, glycyrrhizic acid, glycyrrhetinic acid, camosine, Butylated Hydroxytoluene (BHT) and Butylated Hydroxyanisole (BHA), menthyl anthranilate, cetyl pyridinium chloride, tetrahydrocurmin, vanillin or its derivatives, ergothioneine, melanostatine, sterol esters, idebenone, dehydroacetic acid, yeast extract (e.g., Pitera®), beta glucans, alpha glucans, fatty acids (especially poly-unsaturated fatty acids), zinc pyrithione (ZPT), anti-fungal agents, thiol compounds (e.g., N-acetyl cysteine, glutathione, thioglycolate), other vitamins (vitamin B 12), beta-carotene, ubiquinone, amino acids, their salts, their derivatives, their precursors, and/or combinations thereof. Further description of some of these additional actives is provided below.

The skin and/or hair care actives of the present invention may be useful in skin lightening. Skin lightening may occur through multiple mechanisms including anti-oxidant mechanisms, trypsin inhibition, anti-inflammatory mechanisms, nitric oxide scavenging, tyrosinase inhibition, etc. Thus, compounds which have these mechanisms have the potential to lighten skin.

1. Tetrahydrocurcumin

Compositions of the present invention may comprise a safe and effective amount of tetrahydrocurcumin (THC), its derivatives, such as esters or ethers, and combinations thereof. When present, the composition preferably contains from about 0.01% to about 10%, more preferably from about 0.1% to about 5%, even more preferably from about 0.2% to about 3%, by weight of the composition, of the curcuminoid compound.

Oxygen radicals are produced in the skin in response to many stimuli, such as exposure to UV and irritants. Such radicals are also produced as by-products of normal cell or tissue metabolism. Oxygen radicals can stimulate pigment cells (melanocytes) to increase production of melanin. Curcuminoids (such as THC) have anti-oxidant properties and thus can scavenge oxygen radicals before they stimulate the melanocytes.

The protein tyrosinase is an enzyme involved in the conversion of the amino acid tyrosine to DOPA (dihydroxyphenylalanine) which then is further converted into other intermediates and polymerized into the skin pigment melanin. Partial or complete inhibition of tyrosinase slows or stops, respectively, the formation of melanin, leading to lighter skin color (e.g., reduction in darkness of hyperpigmented spots). Curcuminoids (such as THC) also inhibit tyrosinase.

Other curcuminoids useful in the present invention include curcumin, tetrahydrodemethoxycurcumin, tetrahydrobisdemethoxycurcumin, their derivatives such as esters and ethers, and combinations thereof. Curcuminoids (such as THC) can be of natural or synthetic origin. Preferably, in the present invention, tetrahydrocurcumin is used alone or in combination with tetrahydrodemethoxycurcumin and/or in combination with tetrahydrobisdemethoxycurcumin. Preferred derivatives are esters, particularly the diacetate ester of tetrahydrocurcumin and/or its combination with tetrahydrocurcumin and/or its combination with tetrahydrodemethoxycurcumin and/or its combination with tetrahydrobisdemethoxycurcumin and/or its combination with the esters, particularly the diacetate ester, of tetrahydrodemethoxycurcumin and/or the diacetate ester of tetrahydrobisdemethoxycurcumin.

2. Sugar Amines (Amino Sugars)

The compositions of the present invention may include a safe and effective amount of a sugar amine, which are also known as amino sugars. The sugar amine compounds useful in the present invention are described in PCT Publication WO 02/076423 and U.S. Pat. No. 6,159,485.

When present, the composition preferably contains from about 0.01% to about 15%, more preferably from about 0.1% to about 10%, and even more preferably from about 0.5% to about 5% by weight of the composition, of the sugar amine.

Sugar amines can be synthetic or natural in origin and can be used as pure compounds or mixtures of compounds (e.g., extracts from natural sources or mixtures of synthetic materials). Glucosamine is generally found in many shellfish and can also be derived from fungal sources. As used herein, “sugar amine” includes isomers and tautomers of such and its salts (e.g., HCl salt) and is commercially available from Sigma Chemical Co.

Examples of sugar amines that are useful herein include glucosamine, N-acetyl glucosamine, glucosamine sulfate, mannosamine, N-acetyl mannosamine, galactosamine, N-acetyl galactosamine, their isomers (e.g., stereoisomers), and their salts (e.g., HCl salt). Preferred for use herein are glucosamine, particularly D-glucosamine and N-acetyl glucosamine, particularly N-acetyl-D-glucosamine.

3. Peptides

The compositions of the present invention may contain a safe and effective amount of a peptide, including but not limited to, di-, tri-, tetra-, penta-, and hexa-peptides and derivatives thereof. When present, the compositions preferably contain from about 0.000001% to about 20%, more preferably from about 0.000001% to about 10%, even more preferably from about 0.00001% to about 5%, by weight of the composition.

As used herein, “peptide” refers to peptides containing ten or fewer amino acids and their derivatives, isomers, and complexes with other species such as metal ions (e.g., copper, zinc, manganese, magnesium, and the like). As used herein, peptide refers to both naturally occurring and synthesized peptides. Also useful herein are naturally occurring and commercially available compositions that contain peptides. More preferred peptides are the dipeptide carnosine (beta-ala-his), the tripeptide gly-his-lys, the pentapeptide lys-thr-thr-lys-ser, lipophilic derivatives of peptides, and metal complexes of the above, e.g., copper complex of the tripeptide his-gly-gly (also known as lamin). A preferred dipeptide derivative is palmitoyl-lys-thr. A preferred commercially available tripeptide derivative-containing composition is Biopeptide CL®, which contains 100 ppm of palmitoyl-gly-his-lys and is commercially available from Sederma. A preferred commercially available pentapeptide derivative-containing composition is Matrixyl®, which contains 100 ppm of palmitoyl-lys-thr-thr-lys-ser and is commercially available from Sederma.

4. Hexamidine

The hexamidine compounds useful in the present invention correspond to those of the following chemical structure: 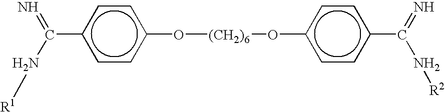
wherein R¹ and R² comprise organic acids (e.g., sulfonic acids, etc.).

When present, the hexamidine preferably comprises from about 0.0001 to about 25%, more preferably from about 0.001 to about 10%, more preferably from about 0.01 to about 5%, and even more preferably from about 0.02 to about 2.5% by weight of the composition.

The topical compositions of the present invention optionally include a safe and effective amount of one or more of hexamidine compounds, its salts, and its derivatives. As used herein, hexamidine derivatives include any isomers and tautomers of hexamidine compounds including but not limited to organic acids and mineral acids, for example sulfonic acid, carboxylic acid etc. Preferably, the hexamidine compounds include hexamidine diisethionate, commercially available as Eleastab® HP 100 from Laboratories Serobiologiques.

5. Cetyl Pyridinium Chloride

The compositions of the present invention may comprise a safe and effective amount of cetyl pyridinium chloride (CPC). Alternate forms of cetyl pyridinium chloride include quaternary compounds in which one or two of the substitutes on the quaternary nitrogen has a carbon chain length (typically alkyl group) from about 8 to about 22, typically from about 10 to about 18 carbon atoms while the remaining substitutes (typically alkyl or benzyl group) have a lower number of carbon atoms, such as from about 1 to about 7 carbon atoms (typically methyl or ethyl groups). The alkyl chain in these alternative forms can be straight chain or branched and can be saturated or unsaturated. Dodecyl trimethyl ammonium bromide, tetradecylpyridinium chloride, cetyl dimethyl benzylammonium chloride, 1-hexadecylpyridinium chloride, dimethyloctadecylbenzyl ammonium chloride, hexadecyltrimethyl ammonium chloride, hexadecyltrimethly ammonium bromide, octadecyltrimethyl ammonium chloride, domiphenbromide, N-tetradecyl-4-ethyl pyridinium chloride, dodecyl dimethyl(2-phenoxyethyl)ammonium bromide, benzyl dimethylstearyl ammonium chloride, quaternized 5-amino-1,3-bis(2-ethyl-hexyl)-5-methyl hexahydropyrimidine, benzalkonium chloride, benzethonium chloride and methyl benzethonium chloride are exemplary of typical quaternary ammonium agents. Other compounds are bis-4-(R-amino)-1-pyridinium alkanes as disclosed in U.S. Pat. No. 4,206,215. The negatively-charged counter ion for these quaternary compounds is typically chloride or bromide, but can alternately be fluoride, iodide, sulfite, sulfate, carbonate, and the like.

Cetyl pyridinium chloride or other quaternary compound may be present in an amount of from about 0.005% to about 10% by weight of the composition, more preferably from about 0.01% to about 5%, more preferably from about 0.05% to about 2%. An unanticipated finding is that cetyl pyridinium chloride is an inhibitor of certain enzymes involved in the pigmentation process, such as tyrosinase. It is believed that other quaternary compounds, besides cetyl pyridinium chloride, also inhibit enzymes involved in the pigmentation process. The protein tyrosinase is an enzyme involved in the conversion of the amino acid tyrosine to DOPA (dihydroxyphenylalanine) which then is further converted into other intermediates and polymerized into the skin pigment melanin. Partial or complete inhibition of tyrosinase slows or stops, respectively, the formation of melanin, leading to lighter skin color (e.g., reduction in darkness of hyperpgimented spots).

6. N-acyl Amino Acid Compounds

The topical compositions of the present invention may comprise a safe and effective amount of one or more N-acyl amino acid compounds. The amino acid can be one of any of the amino acids known in the art. The N-acyl amino acid compounds of the present invention correspond to the formula: 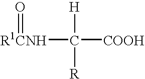
wherein R can be a hydrogen, alkyl (substituted or unsubstituted, branched or straight chain), or a combination of alkyl and aromatic groups. A list of possible side chains of amino acids known in the art are described in Stryer, Biochemistry, 1981, published by W. H. Freeman and Company. R¹ can be C₁ to C₃₀, saturated or unsaturated, straight or branched, substituted or unsubstituted alkyls; substituted or unsubstituted aromatic groups; or mixtures thereof.

Preferably, the N-acyl amino acid compound is selected from the group consisting of N-acyl Phenylalanine, N-acyl Tyrosine, their isomers, their salts, and derivatives thereof. The amino acid can be the D or L isomer or a mixture thereof. N-acyl Phenylalanine corresponds to the following formula: 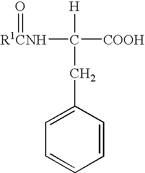
wherein R¹ can be C₁ to C₃₀, saturated or unsaturated, straight or branched, substituted or unsubstituted alkyls; substituted or unsubstituted aromatic groups; or mixtures thereof.

N-acyl Tyrosine corresponds to the following formula: 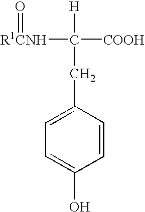
wherein R¹ can be C₁ to C₃₀, saturated or unsaturated, straight or branched, substituted or unsubstituted alkyls; substituted or unsubstituted aromatic groups; or mixtures thereof.

Particularly useful as a topical skin tone evening (lightening or pigmentation reduction) cosmetic agent is N-undecylenoyl-L-phenylalanine. This agent belongs to the broad class of N-acyl Phenylalanine derivatives, with its acyl group being a C11 mono-unsaturated fatty acid moiety and the amino acid being the L-isomer of phenylalanine. N-undecylenoyl-L-phenylalanine corresponds to the following formula:

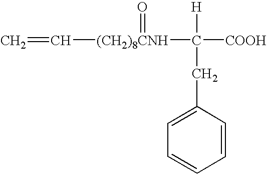

As used herein, N-undecylenoyl-L-phenylalanine is commercially available under the tradename Sepiwhite® from SEPPIC.

When present, the N-acyl amino acid preferably comprises from about 0.0001% to about 25%, more preferably from about 0.001% to about 10%, more preferably from about 0.01% to about 5%, and even more preferably from about 0.02% to about 2.5% by weight of the composition.

7. Glycyrrhizic acid

The compositions of the present invention may include a safe and effective amount of glycyrrhizic acid and/or its salts. When present, the composition preferably contains from about 0.01% to about 10%, more preferably from about 0.05% to about 5%, even more preferably from about 0.1% to about 3%, by weight of the composition, of the glycyrrhizic acid compound. Glycyrrhizic acid is a component of licorice extract.

Glycyrrhizic acid is an anti-inflammatory agent. Inflammatory mediators or cytokines can stimulate pigment cells (melanocytes) to produce melanin. Thus inflammatory conditions such as UV-damage, acne, in-grown hairs, insect bites, scratches, etc. will stimulate what is called post-inflammatory hyperpigmentation. While UV is a primary inducer of pigmentation in all skin types, pigment from the other inflammatory stimuli (acne, etc.) will in particular contribute to skin pigmentation in darker skin individuals (e.g., Hispanic, Asian). Inhibiting inflammation with anti-inflammatory agents will reduce pigmentation.

Glycyrrhizic acid is also believed to be a scavenger of nitric oxide. Nitric oxide (NO) is a stimulator of pigmentation. Use of nitric oxide scavengers (materials that react with nitric oxide to prevent it from stimulating pigment cells) will reduce pigmentation.

Glycyrrhizic acid is also known as glycyrrhizin, glycyrrhizinic acid, or glycyrrhetinic acid glycoside.

8. Glycyrrhetinic acid

The compositions of the present invention may include a safe and effective amount of glycyrrhetinic acid and/or its salts. When present, the composition preferably contains from about 0.01% to about 10%, more preferably from about 0.05% to about 5%, even more preferably from about 0.1% to about 3%, by weight of the composition, of the glycyrrhetinic acid compound. Glycyrrhetinic acid is a component of licorice extract.

Glycyrrhetinic acid is also an anti-inflammatory agent, discussed above in the glycyrrhizic acid section. Structurally, glycyrrhetinic acid is different from glycyrrhizic acid in that glycyrrhetinic acid does not have an attached sugar residue (glycoside). Glycyrrhetinic acid is also known as enoxolone, glycyrrhetic acid, or uralenic acid.

9. Ergothioneine

The compositions of the present invention may comprise a safe and effective amount of ergothioneine. Ergothioneine is preferably present in an amount of from about 0.0001% to about 20% by weight of the composition, more preferably from about 0.001% to about 5% by weight of the composition, even more preferably from about 0.01% to about 1% by weight of the composition. A preferred ergothioneine is Thiotaine® which is a commercial solution of the chemical ergothioneine, commercially available from Barnet Products.

Dermatologically Acceptable Carrier

The topical compositions of the present invention also comprise a dermatologically acceptable carrier for the active materials. The phrase “dermatologically acceptable carrier”, as used herein, means that the carrier is suitable for topical application to the keratinous tissue, has good aesthetic properties, is compatible with the actives of the present invention and any other components, and will not cause any safety or toxicity concerns. A safe and effective amount of carrier is from about 50% to about 99.99%, preferably from about 60% to about 99.9%, more preferably from about 70% to about 98%, and even more preferably from about 80% to about 95% of the composition.

The carrier can be in a wide variety of forms. For example, emulsion carriers, including, but not limited to, oil-in-water, water-in-oil, silicone-in-water, water-in-silicone, water-in-oil-in-water, and oil-in-water-in-silicone emulsions, are useful herein.

Preferred carriers comprise an emulsion such as oil-in-water emulsions and water-in-oil emulsions, e.g., silicone-in-water or water-in-silicone emulsions. As will be understood by the skilled artisan, a given component will distribute primarily into either the water or oil phase, depending on the water solubility/dispensability of the component in the composition. Oil-in-water emulsions are especially preferred.

Emulsions according to the present invention generally contain a solution as described above and a lipid or oil. Lipids and oils may be derived from animals, plants, or petroleum and may be natural or synthetic (i.e., man-made). Preferred emulsions also contain a humectant, such as glycerin. Emulsions will preferably further contain from about 0.1% to about 10%, more preferably from about 0.2% to about 5%, of an emulsifier, based on the weight of the composition. Emulsifiers may be nonionic, anionic or cationic. Suitable emulsifiers are disclosed in, for example, U.S. Pat. No. 3,755,560, U.S. Pat. No. 4,421,769, and McCutcheon's Detergents and Emulsifiers, North American Edition, pages 317-324 (1986).

Suitable emulsions may have a wide range of viscosities, depending on the desired product form. Exemplary low viscosity emulsions, which are preferred, have a viscosity of about 50 centistokes or less, more preferably about 10 centistokes or less, even more preferably about 5 centistokes or less.

The compositions of the present invention can also comprise other dermatologically acceptable topical carriers and can also comprise oral carriers. For example, another topical carrier can be a surfactant-containing cleanser (e.g., bar, shampoo, foaming cleanser, liquid cleanser, body wash, cleansing cloth, and the like). In such a carrier, the surfactant can be anionic, cationic, zwitterionic, nonionic, or mixtures of these. Another topical carrier example is a color cosmetic (lipstick, rouge, eye liner, mascara, foundation, nail polish, and the like). An oral carrier can be a beverage, food item, pill, capsule, powder, caplet, and the like.

Optional Components

The compositions of the present invention may contain a variety of other ingredients that are conventionally used in given product types provided that they do not unacceptably alter the benefits of the invention.

The optional components, when incorporated into the composition, should be suitable for use in contact with human keratinous tissue without undue toxicity, incompatibility, instability, allergic response, and the like within the scope of sound judgment. The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, anti-caking agents, antifoaming agents, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, and thickeners.

The compositions of the present invention may also include the synthetic cationic polymer Polyquaternium-37 (methacryloylethyl trimethyl ammonium chloride homopolymer). This polymer may be added to the compositions as a powder or as a liquid dispersion. This polymer is commercially available under the tradenames Synthalen (3V Sigma), Ultragel 300 (Cosmetic Rheologies Ltd.), Rheocare CTH(E) (Cosmetic Rheologies Ltd.), Salcare SC95 and Salcare SC96 (Ciba Specialty Chemicals).

Other optional components useful in the present invention include those described in U.S. Publication No. 200410175347A1, including desquamation actives, such as salicylic acid and zwitterionic surfactants; soothing and/or healing agents; skin treating agents; skin sensates, astringents, etc. (e.g., clove oil, menthol, camphor, eucalyptus oil, eugenol, menthyl lactate, witch hazel distillate); anti-acne actives, such as resorcinol, sulfur, erythromycin, zinc, dehydroacetic acid; anti-wrinkle actives/anti-atrophy actives; anti-oxidants/radical scavengers, such as tocopherol; chelators, such as furildioxime and derivatives thereof; flavonoids; anti-inflammatory agents; anti-cellulite agents; tanning actives such as dihydroxyacetone; skin lightening agents; antimicrobial and antifungal actives; sunscreen actives; conditioning agents such as glycerol, urea, petrolatum, sucrose polyester, and combinations thereof; thickening agents such as carboxylic acid polymers, crosslinked polyacrylate polymers, polyacrylamide polymers, polysaccharides, gums; water-soluble vitamins; and particulate materials. Compositions of the present invention may contain a safe and effective amount of one or more of the following other actives or ingredients: fatty acids (especially poly-unsaturated fatty acids), glucosamine, zinc pyrithione (ZPT), thiol compounds (e.g., N-acetyl cysteine, glutathione, thioglycolate), other vitamins (e.g., B1, B2, B5,B6, B12, C, D, E, F, K, P), beta-carotene, ubiquinone, idebenone, amino acids, minerals (e.g., Zn, Mn, Mg, Cu, Fe, and Se), hydroxy acids (e.g., alpha-hydroxy acids, alpha-keto acids, and beta-hydroxy acids), kojic aid, arbutin, mulberry extract, exfoliation agents, anti-dandruff agents, and the like.

### Composition Forms

The topical compositions of the subject invention, including but not limited to lotions, milks, mousses, serums, sprays, aerosols, foams, sticks, pencils, gels, creams and ointments, may comprise a dermatologically acceptable emollient. Such compositions preferably contain from about 2% to about 50% of the emollient. As used herein, “emollient” refers to a material useful for the prevention or relief of dryness, as well as for the protection of the skin. A wide variety of suitable emollients are known and may be used herein. Sagarin, Cosmetics, Science and Technology, 2nd Edition, Vol. 1, pp. 32-43 (1972), contains numerous examples of materials suitable as an emollient. A preferred emollient is glycerin. Glycerin is preferably used in an amount of from about 0.001% to about 20%, more preferably from about 0.01% to about 15%, and even more preferably from about 0.1% to about 10% by weight of the composition.

Compositions of this invention useful for cleansing (“cleansers”) are formulated with a suitable carrier (e.g., as described above, and from about 1% to about 90%, by weight of the composition, of a dermatologically acceptable surfactant).

The physical form of the cleansing compositions is not critical. The compositions can be, for example, formulated as toilet bars, liquids, shampoos, bath gels, hair conditioners, hair tonics, pastes, or mousses. Toilet bars are preferred since this is the form of cleansing agent most commonly used to wash the skin. Rinse-off cleansing compositions, such as shampoos, require a delivery system adequate to deposit sufficient levels of actives on the skin and scalp. A preferred delivery system involves the use of insoluble complexes. For a more complete disclosure of such delivery systems, see U.S. Pat. No. 4,835,148.

The compositions of the present invention may also be in the form of cosmetics. Suitable cosmetic forms include, but are not limited to, foundations, lipsticks, rouges, mascaras, and the like. Such cosmetic products may include conventional ingredients such as oils, colorants, pigments, emollients, fragrances, waxes, stabilizers, and the like. Exemplary carriers and such other ingredients which are suitable for use herein are described, for example, in U.S. Pat. No. 6,060,547.

The compositions of the present invention may also be in the form of shave prep products, including, for example, gels, foams, lotions, and creams; and include both aerosol and non-aerosol versions.

### Composition Preparation

The compositions of the present invention are generally prepared by conventional methods such as are known in the art of making topical compositions. Such methods typically involve mixing of the ingredients in one or more steps to a relatively uniform state, with or without heating, cooling, application of vacuum, and the like. The compositions are preferably prepared such as to optimize stability (physical stability, chemical stability, photostability) and/or delivery of the active materials. This optimization may include appropriate pH (e.g., less than 7), exclusion of materials that can complex with the active agent and thus negatively impact stability or delivery (e.g., exclusion of contaminating iron), use of approaches to prevent complex formation (e.g., appropriate dispersing agents or dual compartment packaging), use of appropriate photostability approaches (e.g., incorporation of sunscreen/sunblock, use of opaque packaging), etc.

### Methods for Regulating Keratinous Tissue Condition

The compositions of the present invention are useful for regulating a number of mammalian keratinous tissue conditions. Such regulation of keratinous tissue conditions includes prophylactic and therapeutic regulation. More specifically, such regulating methods are directed to, but are not limited to, thickening keratinous tissue (i.e., building the epidermis and/or dermis and/or subcutaneous layers of the skin and where applicable the keratinous layers of the nail and hair shaft), preventing, retarding, improving, and/or treating uneven skin tone by acting as a lightening or pigmentation reduction cosmetic agent, preventing, retarding, and/or treating atrophy of mammalian skin, softening and/or smoothing lips, hair and nails of a mammal, preventing, retarding, and/or treating itch of mammalian skin, preventing, retarding, and/or treating the appearance of dark under-eye circles and/or puffy eyes, preventing, retarding, and/or treating sallowness of mammalian skin, preventing, retarding, and/or treating sagging (i.e., glycation) of mammalian skin, preventing and/or retarding tanning of mammalian skin, desquamating, exfoliating, and/or increasing turnover in mammalian skin, reducing the size of pores in mammalian skin, regulating oily/shiny appearance of mammalian skin, preventing, retarding, and/or treating hyperpigmentation such as post-inflammatory hyperpigmentation, preventing, retarding, and/or treating the appearance of spider vessels and/or red blotchiness on mammalian skin, preventing, retarding, and/or treating fine lines and wrinkles of mammalian skin, preventing, retarding, and/or treating skin dryness (i.e., roughness, scaling, flaking) and preventing, retarding, and/or treating the appearance of cellulite in mammalian skin. The compositions of the present invention may also be useful in inhibiting hair growth, reducing shaving frequency, improving ease of shaving, decreasing shaving frequency, making hair softer and/or finer, making hair less noticeable, slowing the re-growth of hair, reducing erythema and/or irritation to skin, making skin smoother and/or silkier, and improving the hair removal process.

Regulating keratinous tissue condition involves topically applying to the keratinous tissue a safe and effective amount of a composition of the present invention. The amount of the composition that is applied, the frequency of application and the period of use will vary widely depending upon the level of skin and/or hair care actives and/or other components of a given composition and the level of regulation desired.

In a preferred embodiment, the composition is chronically applied to the skin. By “chronic topical application” is meant continued topical application of the composition over an extended period during the subject's lifetime, preferably for a period of at least about one week, more preferably for a period of at least about one month, even more preferably for at least about three months, even more preferably for at least about six months, and more preferably still for at least about one year. While benefits are obtainable after various maximum periods of use (e.g., five, ten or twenty years), it is preferred that chronic applications continue throughout the subject's lifetime. Typically applications would be on the order of about once per day over such extended periods, however application rates can vary from about once per week up to about three times per day or more.

A wide range of quantities of the compositions of the present invention can be employed to provide a skin appearance and/or feel benefit. Quantities of the present compositions, which are typically applied per application, are in mg composition/cm² skin, from about 0.1 mg/cm² to about 20 mg/cm². A particularly useful application amount is about 0.5 mg/cm² to about 10 mg/cm².

Treating keratinous tissue condition can be practiced, for example, by applying a composition in the form of a skin lotion, clear lotion, milky lotion, cream, gel, foam, ointment, paste, emulsion, spray, aerosol, conditioner, tonic, cosmetic, lipstick, foundation, nail polish, after-shave, roll-on or deodorant stick, powder, oil or the like which is intended to be left on the skin or other keratinous tissue for some aesthetic, prophylactic, therapeutic or other benefit (i.e., a “leave-on” composition). After applying the composition to the keratinous tissue (e.g., skin), it is preferably left on for a period of at least about 15 minutes, more preferably at least about 30 minutes, even more preferably at least about 1 hour, even more preferably for at least several hours, e.g., up to about 12 hours. Any part of the external portion of the face, hair, and/or nails can be treated, (e.g., face, lips, under-eye area, eyelids, scalp, neck, torso, arms, hands, legs, feet, fingernails, toenails, scalp hair, eyelashes, eyebrows, etc.) The composition can be dispensed from a bottle, jar, tube, sachet, pouch, container, tottle, vial, ampule, compact, etc. or can be integrally contained within a delivery form such as a wipe. The application of the present compositions may be done using the palms of the hands and/or fingers. The application may also be done with the aid of a device or implement such as a cotton ball, swab, pad, brush, eye dropper, puff, sponge, wand, wipe, foam, nonwoven substrate, mask, roll-on applicator, stick applicator, applicator pen, spray applicator, atomizer, razor, etc. The active may be contained in a rupturable pouch between two substrates.

In another embodiment, the application of the topical composition is subsequent to a skin treatment such as cleansing, exfoliation or tanning.

Another approach to ensure a continuous exposure of the keratinous tissue to at least a minimum level of the composition is to apply the compound by use of a patch applied, e.g., to the face. Such an approach is particularly useful for problem skin areas needing more intensive treatment (e.g., facial crows feet area, frown lines, under eye area, upper lip, and the like). The patch can be occlusive, semi-occlusive or non-occlusive, and can be adhesive or non-adhesive. The composition can be contained within the patch or be applied to the skin prior to application of the patch. The patch can also include additional actives such as chemical initiators for exothermic reactions such as those described in PCT application WO 9701313, and in U.S. Pat. Nos. 5,821,250, 5,981,547, and 5,972,957 to Wu, et al. The patch can also contain a source of electrical energy (e.g., a battery) to, for example, increase delivery of the composition and active agents (e.g., iontophoresis). The patch is preferably left on the keratinous tissue for a period of at least about 5 minutes, more preferably at least about 15 minutes, more preferably still at least about 30 minutes, even more preferably at least about 1 hour, even more preferably at night as a form of night therapy.

Another approach to enhancing the benefits of the actives is use of a kit or regimen of 2 or 3 or 4 or more products and/or treatment procedures (e.g., exfoliation followed by topical treatment with one or more of the actives of the present invention, depilation of hair followed by topical treatment with one or more of the actives of the present invention, and the like). The various components of a regimen can be used in a short period of time (e.g., within an hour) or spread over a longer time frame within a day (e.g., morning and evening) or over even longer time periods (e.g., one step in the regimen done weekly or monthly and the other steps in the regimen done on a more regular basis, e.g., daily).

Combinations of an oral composition and a topical composition can be packaged together as a kit. In another embodiment, the oral composition and the topical composition are not packaged together as a kit, but potential users of the regimen are informed (e.g., through advertisements, product labeling) that the oral and the topical compositions may be used in conjunction with one another to regulate the condition of keratinous tissue.

The present invention also contemplates the delivery of energy, via a device, to keratinous tissue, either simultaneously and/or sequentially (e.g., within 10 minutes) with application of the topical compositions. The energy delivery device may deliver energy in a variety of forms, including but not limited to energy in the form of light, heat, sound (including ultrasonic waves), magnetic energy, electromagnetic energy (including radio frequency waves and microwaves), mechanical energy (exfoliating or microdermabrasion device), and combinations thereof. The delivery of energy may be continuous, pulsed, modulated, non-modulated, and combinations thereof. In one embodiment, the energy delivery device is hand-held. Alternatively, the energy delivery device is cordless.

The energy may be applied by holding a device within a single area of keratinous tissue, and subsequently moving the device to another area of tissue (or “stamping”). Alternatively, the energy may be applied as the device is continuously moved, or scanned, across the surface of the tissue. The device may be held in substantially continuous contact with the surface of the keratinous tissue, as with laser devices, or may be held at a short distance from the keratinous tissue with the energy directed toward the surface, as with flash lamps.

A temperature change may be simultaneously induced in the keratinous tissue or alternatively, in a compound applied to the surface of the tissue. This temperature change is in addition to any temperature change induced by the delivered energy itself. For example, the keratinous tissue may be slightly warmed prior to delivery of energy, or alternatively, the keratinous tissue may be cooled after delivery of energy.

For energy derived from ultraviolet light sources, the wavelength will generally fall within the UV-A range, from about 315 to about 400 nm (nanometer). For energy derived from visible light sources, the wavelength will generally range from about 400 nm to about 700 nm. For energy derived from infrared (IR) light sources, the wavelength will generally range from about 700 nm to about to about 3000 nm. The amount of energy delivered, or “output fluence,” may be in the range of about 1 J/cm² to about 100 J/cm², where “J” means Joules. For pulsed light sources, the pulse length may range from about 0.001 seconds to about 3 seconds, with an average pulse duration of from about 0.001 seconds to about 1 second. The surface area of keratinous tissue to be covered will vary depending on the application. These and other parameters relevant to delivery of energy depend upon the type of treatment and the type of tissue to be treated, and will appropriately be selected by one of skill in the art.

The present invention also provides articles of commerce that include the personal care compositions described herein, wherein at least one of the personal care composition, packaging for the personal care composition, and advertisement material pertaining to the personal care composition comprises indicia and/or an image which communicates to a consumer that the personal care composition can be used in conjunction with an energy delivery device for regulating the condition of mammalian keratinous tissue.

### EXAMPLES

The following are non-limiting examples of the compositions of the present invention. The examples are given solely for the purpose of illustration and are not to be construed as limitations of the present invention, as many variations thereof are possible without departing from the spirit and scope of the invention, which would be recognized by one of ordinary skill in the art. In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the present invention as described herein.

Content in formulation (g component per 100 g formulation)

| Component | A | B | C | D | E | F |
|---|---|---|---|---|---|---|
| Disodium EDTA | 0.100 | 0.100 | 0.100 | 0.100 | 0.100 | 0.100 |
| Oxynex ® | 2.000 | 2.000 | 2.000 | 2.000 | 2.000 | 2.000 |
| Hexamidine diisethionate | 0.100 | 0 | 0 | 0 | 0 | 0 |
| Tetrahydrocurcumin | 0 | 0.500 | 0 | 0 | 0 | 0 |
| Glycyrrhetinic acid | 0 | 0 | 0.300 | 0 | 0 | 0 |
| Thiotaine ®¹ | 0 | 0 | 0 | 5.000 | 0 | 0 |
| N-undecylenoyl-L-phenylalanine | 0 | 0 | 0 | 0 | 1.000 | 0 |
| N-acetyl glucosamine | 0 | 0 | 0 | 0 | 0 | 2.000 |
| Niacinamide | 5.000 | 5.000 | 5.000 | 5.000 | 5.000 | 5.000 |
| Citric acid | 0.015 | 0 | 0 | 0 | 0 | 0 |
| Isohexadecane | 3.000 | 3.000 | 3.000 | 3.000 | 3.000 | 3.000 |
| Isopropyl isostearate | 1.330 | 1.330 | 1.330 | 1.330 | 1.330 | 1.330 |
| Isopropyl N-laurosylsarcosinate | 0 | 0 | 5.000 | 0 | 0 | 0 |
| Sucrose polycottonseedate | 0.670 | 0.670 | 0.670 | 0.670 | 0.670 | 0.670 |
| Polymethylsilsesquioxane | 0.250 | 0.250 | 0.250 | 0.250 | 0.250 | 0.250 |
| Cetearyl glucoside + cetearyl | 0.200 | 0.200 | 0.200 | 0.200 | 0.200 | 0.200 |
| alcohol | | | | | | |
| Behenyl alcohol | 0.400 | 0.400 | 0.400 | 0.400 | 0.400 | 0.400 |
| Ethylparaben | 0.200 | 0.200 | 0.200 | 0.200 | 0.200 | 0.200 |
| Propylparaben | 0.100 | 0.100 | 0.100 | 0.100 | 0.100 | 0.100 |
| Cetyl alcohol | 0.320 | 0.320 | 0.320 | 0.320 | 0.320 | 0.320 |
| Stearyl alcohol | 0.480 | 0.480 | 0.480 | 0.480 | 0.480 | 0.480 |
| Tocopheryl acetate | 0.500 | 0.500 | 0.500 | 0.500 | 0.500 | 0.500 |
| PEG-100 stearate | 0.100 | 0.100 | 0.100 | 0.100 | 0.100 | 0.100 |
| Glycerin | 7.000 | 7.000 | 7.000 | 7.000 | 7.000 | 7.000 |
| Titanium dioxide | 0.604 | 0.604 | 0.604 | 0.604 | 0.604 | 0.604 |
| Polyacrylamide + C13-14 isoparaffin + | 3.000 | 2.000 | 2.000 | 2.000 | 2.000 | 2.000 |
| laureth-7 | | | | | | |
| Panthenol | 1.000 | 1.000 | 1.000 | 1.000 | 1.000 | 1.000 |
| Benzyl alcohol | 0.400 | 0.400 | 0.400 | 0.400 | 0.400 | 0.400 |
| Dimethicone + dimethiconol | 2.000 | 2.000 | 2.000 | 2.000 | 2.000 | 2.000 |
| Water (to 100 g) | to | to | to | to | to | to |
| | 100 | 100 | 100 | 100 | 100 | 100 |
| TOTAL | 100 | 100 | 100 | 100 | 100 | 100 |

¹Thiotaine is a solution of ergothioneine, and the ergothioneine content is 0.04-0.05%.

Content in formulation (g component per 100 g formulation)

| Component | G | H | I |
|---|---|---|---|
| Disodium EDTA | 0.100 | 0.100 | 0.100 |
| Oxynex ® | 2.000 | 2.000 | 2.000 |
| Cetyl pyridinium chloride | 0.200 | 0 | 0 |
| Pitera ® | 0 | 10 | 0 |
| Ascorbyl glucoside | 0 | 0 | 2.000 |
| Niacinamide | 5.000 | 5.000 | 5.000 |
| Polyquaternium 37 | 0 | 0 | 0 |
| Isohexadecane | 3.000 | 3.000 | 3.000 |
| Isopropyl isostearate | 1.330 | 1.330 | 1.330 |
| Sucrose polycottonseedate | 0.670 | 0.670 | 0.670 |
| Polymethylsilsesquioxane | 0.250 | 0.250 | 0.250 |
| Cetearyl glucoside + cetearyl alcohol | 0.200 | 0.200 | 0.200 |
| Behenyl alcohol | 0.400 | 0.400 | 0.400 |
| Ethylparaben | 0.200 | 0.200 | 0.200 |
| Propylparaben | 0.100 | 0.100 | 0.100 |
| Cetyl alcohol | 0.320 | 0.320 | 0.320 |
| Stearyl alcohol | 0.480 | 0.480 | 0.480 |
| Tocopheryl acetate | 0.500 | 0.500 | 0.500 |
| PEG-100 stearate | 0.100 | 0.100 | 0.100 |
| Glycerin | 7.000 | 7.000 | 7.000 |
| Titanium dioxide | 0.604 | 0.604 | 0.604 |
| Polyacrylamide + C 13-14 isoparaffin + | 2.000 | 2.000 | 2.000 |
| laureth-7 | | | |
| Panthenol | 1.000 | 1.000 | 1.000 |
| Benzyl alcohol | 0.400 | 0.400 | 0.400 |
| Dimethicone + dimethiconol | 2.000 | 2.000 | 2.000 |
| Water (to 100 g) | to 100 | to 100 | to 100 |
| TOTAL | 100 | 100 | 100 |

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All documents cited in the Background, Summary of Preferred Embodiments, and Detailed Description of Illustrative and Preferred Embodiments are, in relevant part, incorporated herein by reference; the citation of any document is not to be construed as an admission that it is prior art with respect to the present invention.

What is claimed is:

1. A personal care composition comprising:
a) diethylhexyl syringylidene malonate;
b) at least one additional skin and/or hair care active selected from the group consisting of tetrahydrocurcumin, sugar amines, peptides, phytosterol, dialkanoyl hydroxyproline, hexamidine compounds, cetyl pyridinium chloride, N-acyl amino acid compounds, hesperedin, mustard seed extract, glycyrrhizic acid, glycyrrhetinic acid, ergothioneine, melanostatine, sterol esters, idebenone, dehydroacetic acid, Licohalcone A, creatine, creatinine, fever few extract, yeast extract, beta glucans, alpha glucans, their salts, their derivatives, their precursors, and/or combinations thereof; and
c) a dermatologically acceptable carrier.

2. The personal care composition of claim 1, wherein the at least one additional skin and/or hair care active is tetrahydrocurcumin.

3. The personal care composition of claim 1, wherein the at least one additional skin and/or hair care active is a sugar amine.

4. The personal care composition of claim 3, wherein the sugar amine comprises N-acetyl glucosamine.

5. The personal care composition of claim 1, wherein the at least one additional skin and/or hair care active is a hexamidine compound.

6. The personal care composition of claim 1, wherein the at least one additional skin and/or hair care active is an N-acyl amino acid compound.

7. The personal care composition of claim 6, wherein the N-acyl amino acid compound is N-undecylenoyl-L-phenylalanine.

8. The personal care composition of claim 1, wherein the at least one additional skin and/or hair care active is glycyrrhetinic acid.

9. The personal care composition of claim 1, wherein the at least one additional skin and/or hair care active is ergothioneine.

10. The personal care composition of claim 1, wherein the at least one additional skin and/or hair care active is a yeast extract.

11. The personal care composition of claim 1, wherein the personal care composition is not marketed as a sunscreen product.

12. The personal care composition of claim 1, further comprising from about 0.001% to about 10%, by weight, of an additional component selected from the group consisting of desquamatory actives, anti-acne actives, wrinkle repair actives, anti-oxidants, radical scavengers, chelators, flavonoids, anti-inflammatory agents, anti-cellulite agents, skin lightening agents, antimicrobial actives, antifungal actives, conditioning agents, thickening agents, water soluble vitamins, oil soluble vitamins, particulate material, topical anesthetics, and combinations thereof.

13. A method for regulating the condition of mammalian keratinous tissue, the method comprising the steps of:
(a) topically applying a personal care composition comprising diethylhexyl syringylidene malonate to a desired area of tissue; and
(b) thereafter applying a second personal care composition comprising a sunscreen active to the desired area of tissue.

14. The method of claim 13, further comprising the step of:
(c) applying energy to the area of tissue via an energy delivery device.

15. The method of claim 14, wherein step (c) is performed simultaneously, at least in part, and/or sequentially with performance of step (a).

16. The method of claim 14, wherein the energy is applied in a form selected from the group consisting of light, heat, sound, magnetic energy, electromagnetic energy, mechanical, and combinations thereof.

17. A method for regulating the condition of mammalian keratinous tissue, the method comprising the steps of:
(a) topically applying a personal care composition comprising diethylhexyl syringylidene malonate to a desired area of tissue; and
(b) applying energy to the area of tissue via an energy delivery device.

18. The method of claim 17, wherein performances of step (a) and step (b) have at least some overlap.

19. The method of claim 17, wherein step (b) is performed within 10 minutes of performing step (a).

20. The method of claim 17, wherein the energy is applied in a form selected from the group consisting of light, heat, sound, magnetic energy, electromagnetic energy, mechanical, and combinations thereof.

21. An article of commerce, comprising:
(a) a personal care composition comprising diethylhexyl syringylidene malonate; and
(b) at least one of packaging for the personal care composition and advertisement material pertaining to the personal care composition comprising indicia and/or an image which communicates that the personal care composition can be used in conjunction with an energy delivery device for regulating the condition of mammalian keratinous tissue.

22. The article of commerce according to claim 21, wherein the energy delivery device transmits energy in a form selected from the group consisting of light, heat, sound, magnetic energy, electromagnetic energy, mechanical, and combinations thereof.
